SEQUENCE LISTING (1 sequence)

SEQ ID NO: 1 - DNA, 10 bp, synthetic construct
  source 1..10: mol_type = other DNA
atatatatat

(19) United States
(12) Patent Application Publication
(10) Pub. No.: US 2025/0154561 A1
(43) Pub. Date: May 15, 2025

(54) Method for detection of single nucleotide variant

(71) Applicant: KAOHSIUNG MEDICAL UNIVERSITY, Kaohsiung City (TW)

(72) Inventors: Chun-Chi Wang, Kaohsiung City (TW); Ke-Peng Lai, Taichung City (TW)

(73) Assignee: KAOHSIUNG MEDICAL UNIVERSITY, Kaohsiung City (TW)

(21) Appl. No.: 18/506,147

(22) Filed: Nov. 10, 2023

(51) Int. Cl. C12Q 1/6827; C12Q 1/6844

(52) U.S. Cl. CPC C12Q 1/6827; C12Q 1/6846

(57) ABSTRACT

The present invention provides a gene detection method for single nucleotide variations. The method involves gene amplification using specific primers, followed by the utilization of a copper nanocluster synthesis reaction to detect the expression of a target gene sequence. This approach facilitates the identification of wild-type nucleotide sequences, single nucleotide variation sequences, or their combinations, thereby offering precise results in gene detection.

This application also contains a Sequence Listing in a computer readable form, the file name is 4155-KMU-SequenceListing, created on Nov. 8, 2023, the size is 2 KB, which is incorporated herein by reference.

### FIELD OF THE INVENTION

The present invention belongs to the field of genetic testing technology, particularly involving a method for detecting single nucleotide variations of target genes in a sample.

### BACKGROUND OF THE INVENTION

Single nucleotide variants (SNVs) play a crucial role in molecular biology and genetic research as they represent small genetic variations in the genome. SNVs include single nucleotide substitutions, insertions, or deletions, and typically involve a single base of DNA. These variations can lead to changes in gene function and are associated with the occurrence of genetic diseases, cancers, and other disorders, or they can impact drug metabolism and responses.

In the past, genetic testing methods for SNVs typically relied on traditional techniques, such as the Sanger sequencing method. In recent years, numerous new testing methods have been developed, including real-time quantitative polymerase chain reaction (real-time qPCR), gel electrophoresis, capillary gel electrophoresis (CGE), multiplex ligation-dependent probe amplification (MLPA), restriction fragment length polymorphism (RFLP), and single-strand conformation polymorphism (SSCP), among others. Despite the advancement of high-throughput sequencing technologies, the detection of SNVs has become faster. However, these methods often require expensive equipment, skilled operational techniques, and a substantial number of reagents or solutions to achieve the detection objectives. Therefore, in this technical field, there is still room for improvement and innovation, necessitating the development of a direct and simple gene testing method for SNVs.

Metal nanoclusters (NCs) are composed of several to several dozen metal atoms. Due to their size approaching the Fermi wavelength of electrons, they exhibit discrete energy levels, leading to strong fluorescence. Among various metal nanoclusters, gold, silver, and copper nanoclusters exhibit excellent performance and are widely applied in biosensors. Moreover, copper nanoclusters, due to their extremely high synthesis efficiency, low cost, and ease of manipulation, have been widely incorporated into biosensors. As a result, this material can serve as a substitute for fluorescence-labeled detection methods, leading to more efficient detection strategies.

The previous literature (Anal. Chem. 2018, 90, 19, 11599-11606) reveals a rapid and simple fluorescence-based genotyping method suitable for the detection of nucleotide variations. This method utilizes streptavidin magnetic beads coupled with biotinylated PCR and restriction fragment release. Through this method, accurate and fast diagnosis of spinal muscular atrophy can be achieved. The advantage of this technique lies in its simplicity and efficiency, and it can be universally applied to the detection of various nucleotide variations. However, this method still requires the labeling of fluorescent molecules for result interpretation. Therefore, developing a rapid and simple non-fluorescence-labeled universal genetic testing method could overcome the current challenges in this field of testing.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flowchart of Embodiment 1. (1) Target gene amplification step; (2) Restriction enzyme digestion step; (3) Magnetic bead separation step; (4) Formation of copper nanoclusters; and (5) Result determination step.

FIG. 2 is a representation of the genotype determination results for Embodiment 1 as detected by a fluorescence spectrophotometer. (1) Wild-type homozygote; (2) Heterozygote of mutant/wild-type; (3) Mutant homozygote; and (4) Blank group.

FIG. 3 illustrates the schematic diagram of the process of Embodiment 2. (1) represents the target gene amplification step; (2) corresponds to the restriction enzyme hydrolysis step; (3) depicts the magnetic bead separation step; (4) outlines the copper nanocluster synthesizing step; and (5) denotes the result determination step.

FIG. 4 shows the calibration curve for determining the proportion of the L858R mutation in Embodiment 2, as detected by a fluorescence spectrophotometer.

FIG. 5 is a flowchart in Embodiment 2, depicting the use of Recombinase Polymerase Amplification (RPA) as the method for gene amplification.

FIG. 6A observes the intensity of relative fluorescence at different concentrations of copper sulfate. FIG. 6B observes the intensity of relative fluorescence at different concentrations of sodium ascorbate.

FIG. 7A shows the results of fluorescence expression using poly-AT as the copper nanocluster synthesis sequence. FIG. 7B presents the double-stranded copper nanocluster synthesis sequence, which includes a genetic sequence: ATATATATAT (SEQ ID NO: 1). FIG. 7C depicts the single-stranded copper nanocluster synthesis sequence.

FIG. 8A shows the results of fluorescence expression using poly-AAT as the copper nanocluster synthesis sequence. FIG. 8B presents the structure of the copper nanocluster synthesis sequence when it is fully paired. FIG. 8C depicts the misaligned structure formed during the pairing of the copper nanocluster synthesis sequence.

FIG. 9 compares the results of fluorescence expression for different sequences used as copper nanocluster synthesis sequences.

FIG. 10 shows the results of direct visual observation of fluorescence expression using poly-AAT as the copper nanocluster synthesis sequence (1: Blank group, 2: Non-target group, 3: Target group).

### DETAILED DESCRIPTION OF THE INVENTION

The present invention offers a solution by providing a universal, rapid, simple, and efficient method for detecting all nucleotide variations identifiable by specific restriction enzymes. This technology holds the efficacy for use in the rapid and accurate diagnosis of genotypes.

The present invention utilizes magnetic beads in conjunction with biotin-labeled primers for gene amplification and restriction enzyme cleavage techniques, aimed at directly detecting nucleotide variations. This method can be completed within a single tube and enables simultaneous testing of multiple samples. It facilitates rapid determination of a patient's genotype and assists in selecting appropriate therapeutic drugs. This rapid, simple, and efficient method is universal and can be used to detect all nucleotide variations that can be recognized by specific restriction enzymes. This technique has the efficacy to be used for quickly and accurately diagnosing genotypes.

According to one aspect of the present invention, a method for detecting single nucleotide variations in a gene, comprising: (a) providing a test sample containing a target gene sequence to be detected, wherein the target gene sequence is a wild-type nucleotide sequence, a single nucleotide variant sequence, or a combination thereof; (b) adding a specific primer pair to perform a gene amplification reaction to produce an amplified product, wherein the amplified product includes the target gene sequence, with one end of the amplified product has a sequence capable of synthesizing copper nanoclusters and the other end has a biotin modification; (c) digesting the amplified product with a restriction enzyme to produce a digested product and an undigested amplified product; wherein the restriction enzyme can only digest one of the wild-type nucleotide sequence or the single nucleotide variant sequence; wherein the digested product includes a digested fragment with the sequence capable of synthesizing copper nanoclusters and a digested fragment with a biotin modification; (d) adding streptavidin-coated magnetic beads to precipitate the undigested amplified product and the biotin-modified digested fragment with a magnet to obtain a supernatant; (e) conducting a copper nanocluster synthesis reaction in the supernatant, wherein more double-stranded DNA template copper nanoclusters are produced when the supernatant contains more digested fragments with the sequence capable of synthesizing copper nanoclusters; and (f) interpreting the results by determining the condition of the target gene sequence based on the amount of double-stranded DNA template copper nanoclusters.

According to the present invention, there is a method for detecting single nucleotide variations in a gene, in which the gene amplification reaction is a molecular biology technique used to increase the number of DNA or RNA molecules in a sample. Among the methods used for gene amplification, the polymerase chain reaction (PCR) is one of the most widely used. PCR is known for its high sensitivity and high specificity, capable of amplifying specific DNA sequences within a short period. This makes PCR a valuable tool in various applications involving DNA analysis, including the detection of single nucleotide variants as described in this invention. Recombinase polymerase amplification (RPA) is a molecular biology technique used for the rapid amplification of nucleic acid sequences. Unlike PCR, which requires a high-temperature condition, RPA can be performed at room temperature or under isothermal conditions. This technique is primarily applied in nucleic acid testing, gene testing, pathogen detection, and other molecular biology fields. RPA functions by utilizing primers or probes to bind specific regions in DNA or RNA. The process then employs recombinase enzymes and DNA polymerase to synthesize new DNA strands. This process is rapid and efficient, typically completed within a few tens of minutes, and does not require expensive equipment or high-temperature conditions.

In one embodiment of the present invention, the gene amplification reaction is polymerase chain reaction (PCR).

In another embodiment of the present invention, the gene amplification reaction is recombinase polymerase amplification (RPA).

According to the present invention, the method for detecting single nucleotide variations in a gene includes the use of a specific primer pair, which includes a forward primer and a reverse primer. The 5′ end of the forward primer has the sequence capable of synthesizing copper nanoclusters and the 5′ end of the reverse primer has a biotin modification.

In another embodiment of the present invention, the 5′ end of the forward primer has a biotin modification and the 5′ end of the reverse primer has the sequence capable of synthesizing copper nanoclusters.

Currently, there are various online tools and software available for primer design. These tools can generate specific primers for the target gene sequence based on specific requirements, suitable for applications such as PCR, RPA, qPCR, gene testing, etc. Examples include online tools like Primer3, NCBI Primer-BLAST, and others. Those skilled in the art can design primer pairs based on the principles of primer design and using these primer design tools.

According to the present invention, the method for detecting single nucleotide variations in a gene, in which the sequence capable of synthesizing copper nanoclusters is an AT repeat sequence or an AAT repeat sequence.

In one embodiment, the sequence suitable for synthesizing copper nanoclusters is an AAT repeat sequence, and the AAT repeat sequence significantly reduces signal interference.

In another embodiment, the AAT repeat sequence can be AAT, ATA, TAA, TTA, TAT, or ATT repeat sequences.

In another embodiment, the AAT repeat sequence can be the complementary sequence of AAT, ATA, or TAA, where the complementary sequence can be TTA, TAT, and ATT repeat sequence.

In another embodiment, the AAT repeat sequence is a repeat sequence of AAT.

In another embodiment, the AAT repeat sequence is from 10 to 20 AAT repeats.

In another embodiment, the AAT repeat sequence consists of 16 AAT repeats.

In general, the primer sequence used for gene amplification is between 18 to 30 nucleotides in length, with variations based on specific experimental requirements. The final primer length should depend on specific experimental requirements, including the GC content of the target sequence, the expected size of the amplification product, and the conditions of the amplification reaction used. Therefore, the length of the sequence suitable for synthesizing copper nanoclusters at the 5′ end will vary based on the length of the primer sequence for that experiment.

In one embodiment, the primer has a length of 28-50 nucleotides.

According to the present invention, the method for detecting single nucleotide variations in a gene, the selection of the restriction enzyme is based on comparing the wild-type nucleotide sequence and the single nucleotide variant sequence in the target gene sequence, confirming that the chosen restriction enzyme can only recognize and cleavage one of the aforementioned sequences.

Currently, there are various online tools and software available that can assist in finding the appropriate restriction enzyme in line with this principle. By providing potential cutting sites and relevant information, these tools can perform simple validation tests to confirm whether the chosen restriction enzyme meets the requirements of their experimental design.

According to the present invention, the method for detecting single nucleotide variations in a gene involves interpreting results through direct observation to obtain a qualitative outcome. This qualitative result is then used to determine whether the target gene sequence is a wild-type nucleotide sequence or a single nucleotide variant sequence.

In one embodiment, the result interpretation is done by visually observing the color intensity of the supernatant. In another embodiment, the result interpretation is done by visually observing the color intensity of the supernatant under ultraviolet light.

According to the present invention, the method for detecting single nucleotide variations in a gene, the result interpretation is obtained through fluorescence analysis using a fluorescence spectrometer to obtain a quantitative result.

In one embodiment, the quantitative result can be used to determine whether the target gene is likely a wild-type homozygote, a mutant homozygote, or a mutant/wild-type heterozygote.

### Example

The following describes the method of this invention. Although specific embodiments are referenced in this description, it is only for demonstrative purposes and should not be construed as limiting the invention to these embodiments. On the contrary, the spirit and scope of this invention are intended to encompass alternatives, modifications, and equivalents. Therefore, this specification and illustrations should be considered illustrative in nature and not restrictive.

In this specification, numerous specific details are provided to facilitate a thorough understanding of the invention. However, it is apparent to those skilled in the art that the invention can be practiced without these specific details. In some instances, well-known methods, procedures, and materials have not been described in detail to avoid obscuring the essential features of the invention.

### Embodiment 1

Chronic myeloid leukemia (CML) is a disease caused by the fusion of the BCR-ABL1 genes. This fusion gene leads to abnormally enhanced tyrosine kinase activity, which in turn causes excessive cell growth. Therefore, tyrosine kinase inhibitors are often used as the first-line treatment. However, some patients may develop the T315I mutation, which alters the structure of the tyrosine kinase, resulting in resistance to most tyrosine kinase inhibitors. Hence, detecting the T315I mutation in the BCR-ABL1 gene can be crucial for determining appropriate drug therapy.

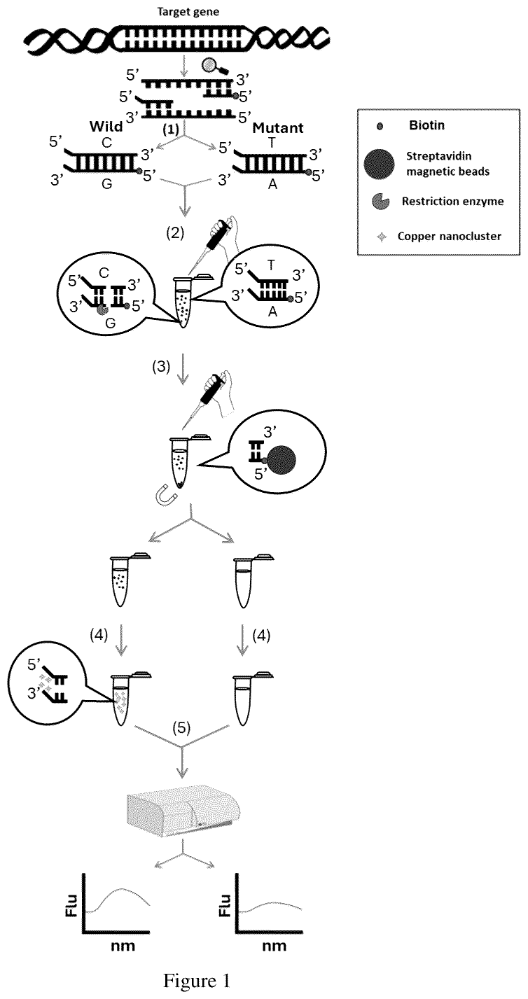

The invention proposes a simple and direct method for detecting the T315I mutation, with the process as illustrated in FIG. 1. The steps include: (1) Amplification of the target: First, DNA samples are subjected to PCR amplification. The forward primer is designed with a copper nanocluster synthesis sequence at the 5′ end, and the reverse primer has a biotin-modification at the 5′ end, primarily for binding with streptavidin-coated magnetic beads; (2) Restriction enzyme digestion reaction: Subsequently, BtsIMutI restriction enzyme is added to recognize and digest the wild-type sequence in the PCR product. The T315I single nucleotide variant (C>T) cannot be digested by BtsIMutI restriction enzyme; (3) Streptavidin-coated magnetic bead binding: After restriction enzyme digestion, streptavidin-coated magnetic beads are added to separate the biotin-guided DNA fragments. As the wild-type sequence can be digested by BtsIMutI restriction enzyme, the digested double-stranded DNA will be released into the supernatant after magnetic bead precipitation. However, the mutant (C>T) sequence cannot be hydrolyzed by the restriction enzyme, so double-stranded DNA is not released; (4) Synthesizing of copper nanoclusters: 30 μL of supernatant is taken, and copper ions and a reducing agent are added to synthesis copper nanoclusters on the double-stranded DNA template; and (5) Result determination: A fluorescence spectrometer is used for detection. The higher fluorescence intensity indicates a higher proportion of the wild-type gene.

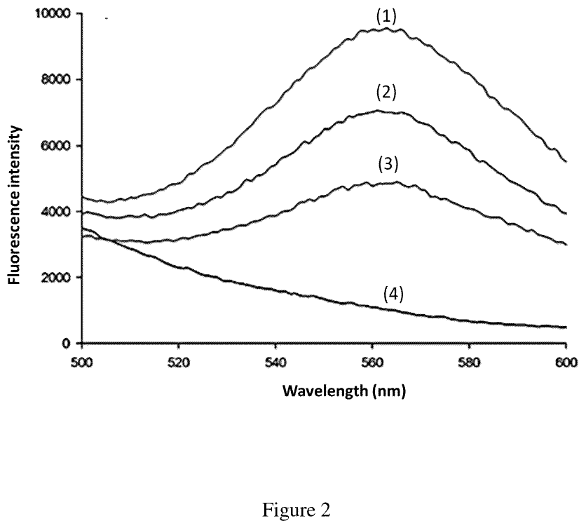

After optimizing this technique, it is applied to detect different genotypic samples, including wild-type homozygotes, mutant homozygotes, and mutant/wild-type heterozygotes. FIG. 2 presents the relevant data, demonstrating that by using the fluorescence intensity of double-stranded DNA template copper nanoclusters as a fluorescence marker, it is possible to easily distinguish between wild-type genotypes and mutant genotypes. Furthermore, in the analysis of mutant/wild-type heterozygotes, the fluorescence intensity is lower than that of the wild-type but higher than that of the mutant.

### Embodiment 2

In the diagnosis and treatment of non-small cell lung cancer (NSCLC), mutations in the epidermal growth factor receptor (EGFR) gene play a key role, particularly as the mutant type shows a good response to targeted drugs, such as tyrosine kinase inhibitors. Therefore, in lung cancer patients, it is necessary to test the epidermal growth factor receptor gene after diagnosis to determine the first-line treatment medication and subsequently assess the prognosis and the content of cancer cells. Among all EGFR gene mutations, exon 21 (L858R) accounts for 45% of the mutations. The mutation site is characterized by a single nucleotide variant where thymine is replaced by guanine. Therefore, a simple and rapid test for the L858R mutation is crucial for NSCLC patients to receive early and precise medication.

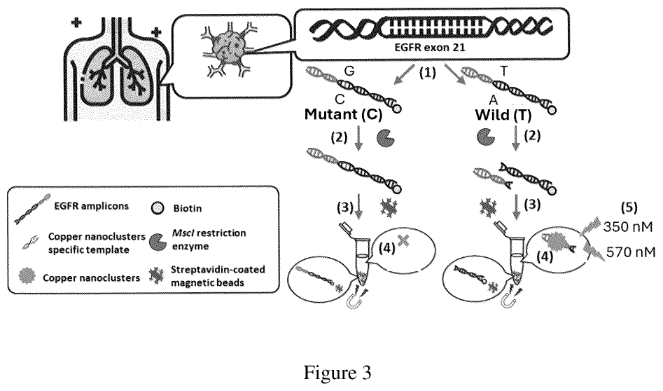

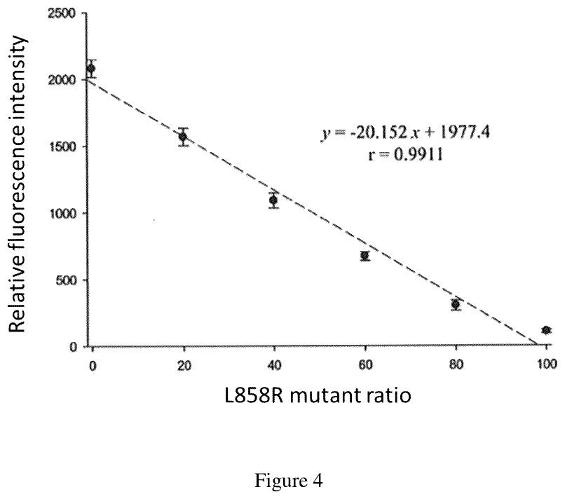

The invention utilizes magnetic beads in conjunction with restriction enzymes, allowing for the quantification of the EGFR gene through fluorescence detection. The process is illustrated in FIG. 3. Firstly, a pair of specific primers is designed to amplify a fragment of the EGFR gene by PCR, which can amplify both the wild-type genotype and the L858R mutant genotype of the EGFR gene. The forward primer is tagged at the 5′ end with a sequence conducive to the synthesis of copper nanoclusters (poly-AAT), while the 5′ end of the reverse primer is modified with biotin. After the PCR reaction is complete, the Msel restriction enzyme is used to recognize the amplified EGFR gene. If the amplification product is the wild-type sequence, it will be cleaved by the restriction enzyme. If the amplification product is a mutant genotype fragment, it will not be cleaved by the restriction enzyme. Consequently, the supernatant will not contain fragments with the sequence (poly-AAT) conducive to the synthesis of copper nanoclusters. Next, streptavidin-coated magnetic beads are added to the solution. The beads will recognize the gene amplification fragments labeled with biotin and capture them. Subsequently, magnetic precipitation is performed using a magnet to separate the beads. Finally, fluorescence analysis is conducted on the supernatant using a fluorescence spectrometer. When the sample is of the wild-type EGFR gene, a strong fluorescence signal can be detected; If the sample contains the L858R mutant type gene, the fluorescence signal will decrease in proportion to its percentage, achieving the purpose of quantification. Through this method, the proportion of the L858R mutation in the tumor can be quantified simply and rapidly (FIG. 4).

### Embodiment 3: Recombinase Polymerase Amplification (RPA) as a Method for Gene Amplification

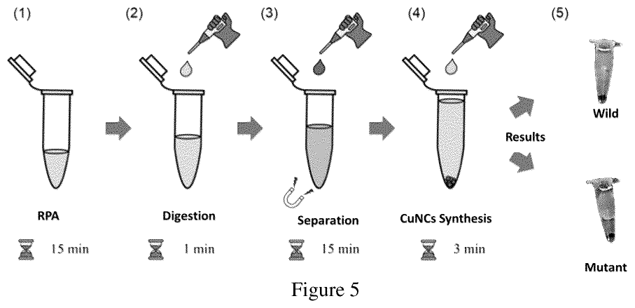

First, the obtained human DNA is subjected to the RPA, an isothermal gene amplification method. After amplification, the desired gene fragments are produced, with each end of the product carrying sequences for the synthesis of copper nanoclusters and being modified with biotin. Then, a restriction enzyme that can recognize the target gene sequence is added. At this stage, the presence of polyethylene glycol in the isothermal gene amplification reagents reduces the reaction time of the restriction enzyme, thereby shortening the reaction duration from 2 hours to just a few minutes. After cutting, the biotin within the fragments is captured using magnetic beads coated with streptavidin on their surface, and these are then precipitated to the bottom under the influence of magnetic force. If the fragments contain the target sequence, then the cleavage fragments will remain in the supernatant. By synthesizing copper nanoclusters in the supernatant, it's possible to determine the quantity of fragments that have been cut, and thus infer the patient's genotype and its proportion. Using the RPA method for gene amplification as a replacement for PCR, the entire reaction can be conducted in a single tube at 37° C. to achieve the desired detection effect. Moreover, the detection time is such that the results can be obtained within 30 minutes (FIG. 5).

After cleavage, biotin in the fragment is captured by magnetic beads coated with streptavidin on the surface and precipitated to the bottom under the influence of magnetic force. If the fragments contain the target sequence, the cleaved fragments will remain in the supernatant. By synthesizing copper nanoclusters in the supernatant, we can determine how many fragments have been cleaved, and thus infer the patient's genotype types and proportions. Using the RPA method for gene amplification as a replacement for PCR, the entire reaction can be conducted in a single tube at 37° C. to achieve the desired detection effect. Moreover, the reaction time is such that the results can be obtained within 30 minutes (FIG. 5).

### Embodiment 4: Optimization of Copper Nanocluster Synthesis

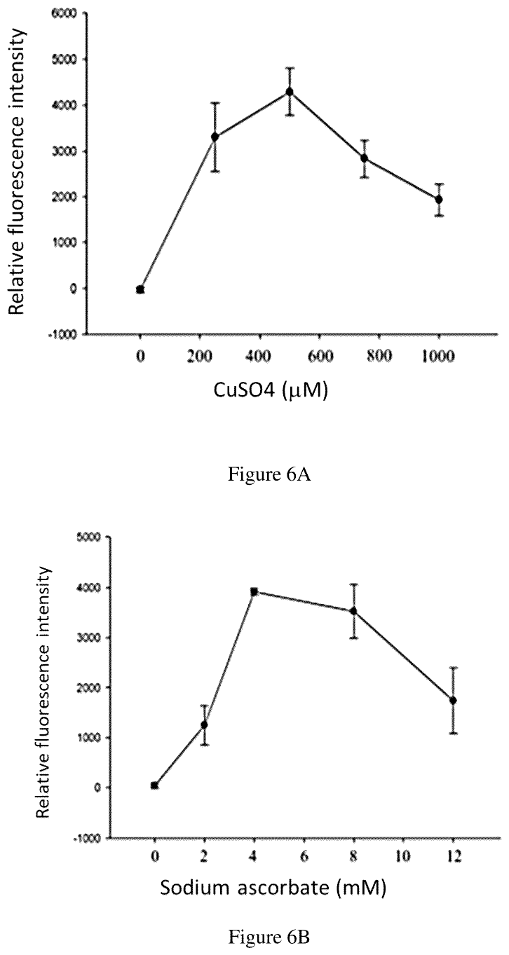

Copper nanoclusters are synthesized in a 200 μL solution, which includes 30 μL of supernatant, a 3-(N-morpholino) propanesulfonic acid (MOPS) buffer (comprising 10 mM MOPS, 150 mM NaCl, with a pH 7.4), and varying concentrations of copper 0, 250, 500, 750, or 1000 μM copper sulfate (CuSO4), and 0, 2, 4, 8, or 12 mM sodium ascorbate. These components are mixed and subjected to a centrifugation process. After a reaction time of 1 minute, the reagents are analyzed using fluorescence spectrophotometry (FIG. 6A and FIG. 6B).

### Embodiment 5: Novel Sequence for Copper Nanocluster Synthesis

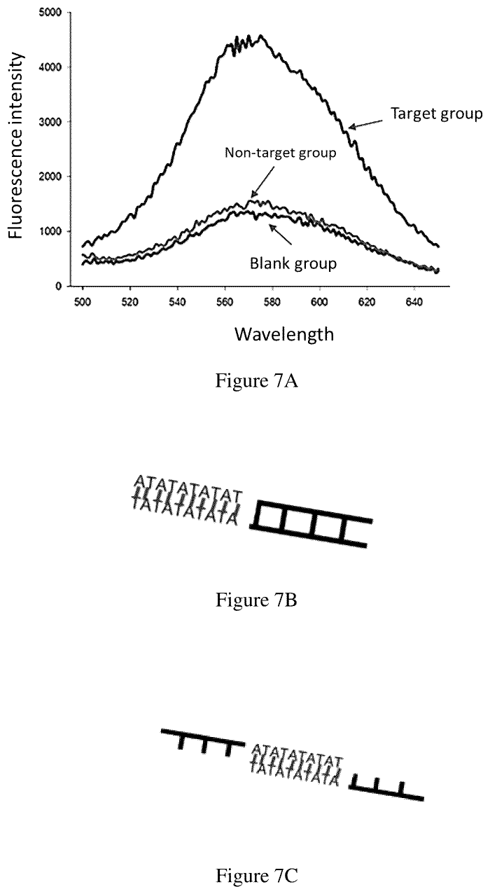

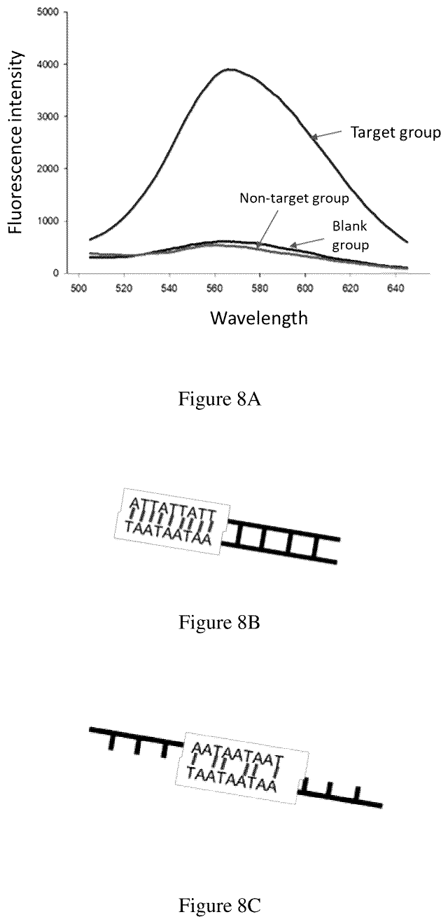

Previous methods for copper nanocluster synthesis have had poor selectivity and significant signal interference issues. FIG. 7A shows the fluorescence performance results using poly-AT as the sequence of copper nanocluster synthesis. Previous research has revealed that the most effective sequence for the copper nanocluster synthesis is a double-stranded poly-AT sequence, which exhibits high synthesis efficiency and strong fluorescence. Consequently, when gene amplification is performed through PCR, the successful gene amplification group produces double-stranded AT sequences (FIG. 7B). In the group where gene amplification is unsuccessful (FIG. 7C), single-stranded primer AT sequences also combine to form double-stranded AT sequences, resulting in non-specific fluorescence interference (FIG. 7A, blank group). This invention has developed a new synthesis sequence that significantly reduces signal interference. FIG. 8A shows the results of fluorescence expression using poly-AAT as the sequence of copper nanocluster synthesis. In the group where gene amplification is successful, complete pairing occurs (FIG. 8B). However, the poly-AAT sequence designed in this invention, when it self-associates, forms a mismatched structure (FIG. 8C). As a result, this design can reduce non-specific fluorescence interference (FIG. 8A, blank group). In this embodiment, the poly-AAT sequence is designed at the 5′ end of the forward primer, including the AAT repeat sequence, and can also include ATA and TAA repeat sequences. Additionally, the reverse of the poly-AAT sequence is the poly-TTA sequence, which includes the TTA repeat sequence, TAT repeat sequence, and ATT repeat sequence. The aforementioned repeat sequences can form similar double-stranded DNA structures and are capable of forming the optimal copper nanocluster synthesis sequence as revealed in this invention. This can reduce the non-specific fluorescence interference. Furthermore, the aforementioned repeat sequences can also be designed at the 5′ end of the reverse primer, achieving the same effect.

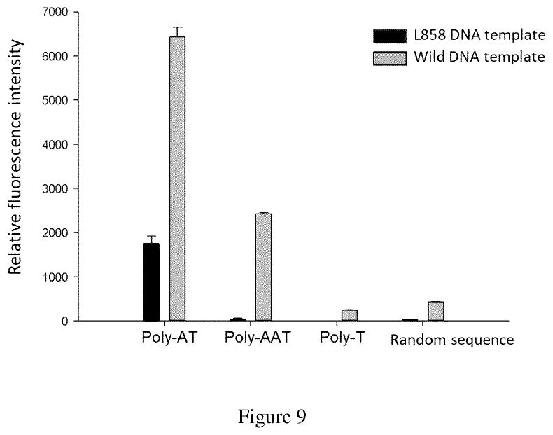

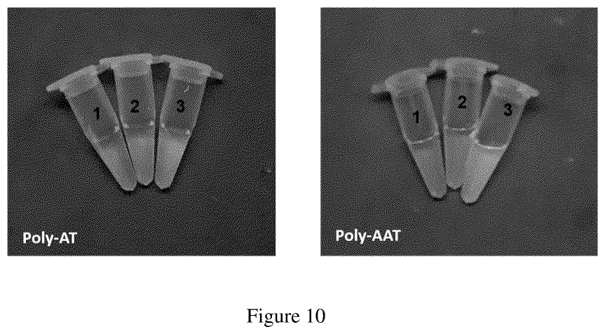

In a comparison with sequences of the same length such as poly-AT, poly-T, and random sequences, the results showed that poly-AT had the strongest fluorescence, but also the highest interference. Poly-T and random sequences had lower interference but also lower fluorescence intensity. By contrast, poly-AAT, although not the strongest in fluorescence, had very low background fluorescence interference (FIG. 9). Therefore, the difference in fluorescence can be distinguished by the naked eye, allowing for the identification of wild-type and mutant genotypes without the need for instrumentation (FIG. 10).

What is claimed is:

1. A method for detecting single nucleotide variants in a gene, comprising:
(a) providing a test sample containing a target gene sequence to be detected, wherein the target gene sequence is a wild-type nucleotide sequence, a single nucleotide variant sequence, or a combination thereof;
(b) adding a specific primer pair to perform a gene amplification reaction to produce an amplified product, wherein the amplified product includes the target gene sequence, with one end of the amplified product has a sequence capable of synthesizing copper nanoclusters and the other end has a biotin modification;
(c) digesting the amplified product with a restriction enzyme to produce a digested product and an undigested amplified product; wherein the restriction enzyme can only digest one of the wild-type nucleotide sequence or the single nucleotide variant sequence; wherein the digested product includes a digested fragment with the sequence capable of synthesizing copper nanoclusters and a digested fragment with a biotin modification;
(d) adding streptavidin-coated magnetic beads to precipitate the undigested amplified product and the biotin-modified digested fragment with a magnet to obtain a supernatant;
(e) conducting a copper nanocluster synthesis reaction in the supernatant, wherein more double-stranded DNA template copper nanoclusters are produced when the supernatant contains more digested fragments with the sequence capable of synthesizing copper nanoclusters; and
(f) interpreting the results by determining the condition of the target gene sequence based on the amount of double-stranded DNA template copper nanoclusters.

2. The method of claim 1, wherein the gene amplification reaction is recombinase polymerase amplification.

3. The method of claim 1, wherein the gene amplification reaction is polymerase chain reaction.

4. The method of claim 1, wherein the specific primer pair includes a forward primer and a reverse primer, wherein the 5′ end of the forward primer has the sequence capable of synthesizing copper nanoclusters and the 5′ end of the reverse primer has a biotin modification, or the 5′ end of the forward primer has a biotin modification and the 5′ end of the reverse primer has the sequence capable of synthesizing copper nanoclusters.

5. The method of claim 1, wherein the sequence capable of synthesizing copper nanoclusters is an AT repeat sequence or an AAT repeat sequence.

6. The method of claim 5, wherein the sequence capable of synthesizing copper nanoclusters is an AAT repeat sequence.

7. The method of claim 6, wherein the AAT repeat sequence can be AAT, ATA, TAA, TTA, TAT, and ATT repeat sequences.

8. The method of claim 6, wherein the result interpretation is done by direct observation to obtain a qualitative result, wherein the qualitative result is used to determine whether the target gene sequence is a wild-type nucleotide sequence or a single nucleotide variant sequence.

9. The method of claim 1, wherein the result interpretation is done by fluorescence analysis using a fluorescence spectrometer to obtain a quantitative result.

10. The method of claim 9, wherein the quantitative result is used to determine the proportion of mutant/wild-type heterozygotes in the target gene sequence.
